(19) United States
(12) Patent Application Publication
(10) Pub. No.: US 2007/0219454 A1
(43) Pub. Date: Sep. 20, 2007

(54) ECG method and system for optimal cardiac disease detection

(76) Inventors: J. James Guzzetta, El Sobrante, CA (US); Steven B. Wolff, Point Roberts, WA (US)

Correspondence Address: J. JAMES GUZZETTA, 4176 SANTA RITA ROAD, EL SOBRANTE, CA, 94803, US

(21) Appl. No.: 11/713,334

(22) Filed: Mar. 2, 2007

Related U.S. Application Data
(60) Provisional application No. 60/755,185, filed on Mar. 2, 2006.

(51) Int. Cl. A61B 5/04

(52) U.S. Cl. 600/509

(57) ABSTRACT

Methods for determining the probability of cardiac disease indicators by utilizing an optimal lead set to measure electrocardiographic data to find the measurement extrema over an optimum portion of the Thorax. The optimal lead topology is designed to produce estimates of total thoracic electrocardiographic information, low noise and errors within the constraints imposed by the measured leads' associated constraint set, which include disease targets. Importantly an optimal electrode topology and measured lead set is deemed optimal when the estimated lead topology provides the lowest global estimation errors. An optimum electrode topology is one that places the electrodes in arbitrary, but optimal, positions on the Thorax and not in a grid like manner (such as used by a BSPM vest electrode array) nor necessarily in those positions used in current practice such as for standard 12 lead or EASI leads.

### CROSS REFERENCE TO RELATED APPLICATIONS

We claim benefit from the U.S. Provisional Application No. 60/755,185 with filing date of Mar. 2, 2006 (Originally submitted Dec. 30, 2005 and rejected for lack of drawings which were faxed to USPTO on Mar. 2, 2006 and then accepted for the filing date of Mar. 2, 2006).

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON COMPACT DISC

Not Applicable

### SEQUENCE LISTING

Not Applicable

### BACKGROUND OF THE INVENTION

1. Field of the Invention

The present invention relates to cardiac electrocardiographic based diagnosis, optimal ECG lead sets, matrix based lead estimation, low noise estimation, electrocardiographic measurements made over the thorax, and reduced lead systems.

2. Background of the Invention

Current ECG methods have many limitations. One is the use of short (10 sec-30 sec) and/or periodic testing, which can be far too short to capture a disease event. Another is that the uses of Standard 12 lead configurations are cumbersome to place and wear. Reduced-lead configurations significantly sacrifice both ECG sensitivity and specificity. Most ECG measurements that are required for accurate Ischemia diagnosis require accurate and stable ST and T measurements, which are usually taken only in a resting electrocardiogram.

Standard 12 leads have the additional limitation that they can be mechanically cumbersome and not usually used in ambulatory, hospital, EMS, and surgical procedures. As a more wearable alternative, various reduced-, and alternative, lead systems have been developed. The most famous is the EASI system.

All the alternative and reduced lead, as well as 12-lead systems are not optimal for capturing the total thoracic ECG data, i.e., there is considerable redundancy in the 12 lead ECG and most reduced lead systems are based on simplicity of application and not optimized for signal capture.

It is known that a large number of electrodes (such as 32 to 192 electrodes used in body surface potential mapping BSPM) can capture total thoracic ECG information whereas standard 12 lead systems, and of course sub-optimal reduced lead systems, always have had ‘blind spots’ for various Ischemic events.

Some practitioners have stopped using EASI or 3-5 lead hospital lead systems because they often miss detecting events.

In addition most ambulatory ECG telemetry performed today uses event type (“loop”) monitors which employ at most two ECG leads in order to capture patient initiated arrhythmic events over extended times while maintaining patient comfort. However these systems do not perform well for capturing Ischemic events, largely a result of inadequate spatial sampling on the thorax.

Many researchers have verified the promise of BSPM systems. The following are two citations thereof:

“BSPM has also been applied to identify the optimal recording sites to detect ischemia-induced ST deviations in patients representing with acute MI. Of these six optimal leads, identified by use of discriminate index analysis, five were outside the standard precordial leads V1-V6 of 12-lead ECG These findings thus imply that improved detection of ischemic changes can be achieved by BSPM.—Kornreich et al., 1993

“BSPM has demonstrated superior sensitivity to 12-lead ECG (88% vs. 38%) while maintaining good specificity (75% vs. 81%) in detection of acute myocardial infarcts”—Menown et al., 2001

However the promise of improved diagnostic capability by large electrode systems such as BSPM systems have been foreshadowed by the cumbersome nature of the electrode systems. This makes them impractical for a wide variety of ECG monitoring and diagnostic systems.

The goal of the invention described herein is to define and utilize optimal electrode arrays for cardiac disease diagnosis.

### DESCRIPTION OF RELATED ART

The following patents may be relevant to the subject matter of the present invention, and their full disclosures incorporated by reference with our comments and comparison to our invention disclosed herein:

Schreck in U.S. Pat. No. 6,901,285 and 20030216655 “System and method for synthesizing leads of an electrocardiogram” discloses a system for synthesizing leads from the I, II, aVf leads and claims that his method will cover 100% of AMIs. However the 100% detection claim is relative only to the sensitivity of the standard 12 lead and not in relation to the total Thoracic ECG information that is available. The methods cited include the use of a covariance matrix to calculate final lead data or lead data for temporary evaluation and optimization. Optimization methods disclosed by Schreck include abstract factor analysis (“AFA”) and Simplex Optimization.

The application area for this patent is for ECG diagnostics, specifically for emergency cardiac triage.

In contrast the invention herein discloses a method to achieve a much greater sensitivity than what Schreck claims. Since Schreck is deriving/synthesizing the leads from a subset of the standard 12 lead system, he is missing the point that Ischemia extrema may be located in regions not detected or even hinted at by the 12 leads including the I, II, aVf leads which are the basis of his claims. The important point here is that a full BSPM 192 lead set, for example, can see distributions that are not detected (below “clinical threshold” for ST changes) or even hinted at when using the standard 12 lead ECG.

Additionally our invention herein discloses methods for utilizing an optimal lead set to measure ECG data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems, used for input, which may have from a few leads to 100s of uni-polar leads where 4 to 24 leads is typical and output is for 4 to 192 leads where 4 to 32 leads is typical.

In sum Schreck discloses a methodology to estimate the electrophysiological potentials on the body surface from specifically and only the I, II, aVf leads, whereas the invention disclosed herein utilizes a optimal lead set of arbitrary topology for multiple disease detection and provides superior Ischemia detection performance.

Groenewegen, et al. 20040138574 “Methods and apparatus for enhancing diagnosis of myocardial infarctions” discloses the use of a vest type electrode array for collecting 30 to 130 leads of BSPM data which is then analyzed and the results compared to a data base of many patients' data to determine the probability and location of a myocardial infarctions or ischemia.

The application area for this patent is for localization of myocardial infarction or ischemia during a cardiac procedure.

In contrast the invention herein discloses methods for utilizing an arbitrary electrode topology rather than a vest topology as does Groenewegen.

Further Groenewegen compares measured data to the data in a database of patients whereas this invention discloses the use of a covariance matrix optimized utilizing ECG data from diseased patients. The covariance matrix derived therefore has the patient data subsumed within the matrix rather than as a separate and large database.

Additionally our invention herein discloses methods for utilizing an optimal lead set to measure ECG data, find the measurement extrema over an optimum portion of the Thorax, and to determine probabilities of disease indicators. Further our invention discloses an optimum matrix which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Groenewegen discloses a 30 to 130 electrode BSPM vest based system for the measurement of electrophysiological potentials on the body surface whereas the invention disclosed herein utilizes a optimal lead set of arbitrary topology for multiple disease detection.

Potse, et al. 20020038093 “Continuous localization and guided treatment of cardiac arrhythmias” discloses the use of a full vest electrode array for collecting 30 to 130 leads of BSPM data which is then analyzed and the results compared to a data base of may patients to determine the probability and location of arrhythmias during surgical procedure.

The application area for this patent is for localization during cardiac EP procedure.

In contrast the invention herein discloses methods for utilizing an arbitrary electrode topology rather than a vest topology as does Potse. In addition Potse discloses a catheter based electrode system for within patient localization.

Further Potse compares various data to the data in a database of patients whereas this invention discloses the use of a covariance matrix optimized utilizing ECG data from diseased patients. The covariance matrix derived therefore has the patient data subsumed within the matrix rather than as a separate and large database.

In sum Potse discloses a 30 to 130 electrode BSPM vest based system and a within patient catheter for the measurement of electrophysiological potentials on the body surface and within the body for localization during cardiac EP procedure whereas the invention disclosed herein utilizes a optimal lead set of arbitrary topology for multiple disease detection

Rudy in U.S. Pat. Nos. 6,772,004, 6,975,900, and 20020128565, 20030120163 “System and methods for noninvasive electrocardiographic imaging (ECGI) using generalized minimum residual (GMRes)” discloses method and system for computing epicardial surface electric potentials based on measured body surface electric potentials for the purpose of performing an EP procedure on a patient's heart. The methods and systems include representing at least one geometric relationship between at least one body surface electric potential measuring system and the epicardial surface as a multidimensional matrix, estimating an inverse of the multidimensional matrix based on a Generalized Minimum Residual (GMRes) method. Electrical potentials are measured on the body surface via an electrode vest, and a body surface potential map is generated. A matrix of transformation based on the geometry of the torso, the heart, locations of electrodes, and position of the heart within the torso is also determined with the aid of a processor, and a geometry-determining device. The electrical potential distribution over the epicardial surface of the heart is then determined based on a regularized matrix of transformation, and the body surface potential map. Using the epicardial potential distributions, epicardial electrogram, isochronal are also reconstructed, and displayed via an output device.

The application area for this patent is for ECG diagnostics, specifically for imaging epicardial surface electric potentials, perhaps for EP procedure guidance.

In contrast the invention herein discloses methods for utilizing an optimal lead set to measure ECG body surface data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Rudy discloses a system for estimation of the electrophysiological potentials on the epicardial surface for EP procedures whereas the invention disclosed herein utilizes a body surface optimal lead set of arbitrary topology for multiple disease detection.

Anderson et al in U.S. Pat. Nos. 6,721,593, 6,778,851, 20020029001, 20020055683, and 20020062087 “Apparatus for body surface mapping” discloses a BSPM method for capturing at least 20 electrodes, computing the standard ST and QRS parameters and determining the condition of the patient's heart using a binary decision tree algorithm.

20020062087 specifies a means for calculating and displaying in graphical form the variation in position with respect to time of at least one characteristic of the sampled values which varies in position in a plane containing the electrodes. Also a characteristic is displayed as projections of the trajectory onto two planes perpendicular to each other and to the plane containing the electrodes.

20020055683 and U.S. Pat. No. 6,778,851 determines the condition of the patient's heart using a binary decision tree algorithm, such algorithm having a plurality of decision nodes each of which makes a decision based upon the value(s) of a respective subset of the parameters, the decision criterion of at least one of the said decision nodes being modified according to a measured value of at least one parameter not of the respective subset.

20020029001 determine the condition of the human heart by comparing the cardiac vectors derived from the ST T and ST60 map vectors with the cardiac vector derived from the QRS map vector.

U.S. Pat. No. 6,721,593 provides for body surface mapping with a two-dimensional array of at least 20 electrodes. Also a means for calculating and displaying in graphical form the variation in position with respect to time of at least one morphological feature of the isopotential maps represented by the plurality of sets of sampled values, wherein the at least one morphological feature is displayed as a projection of a trajectory of the morphological feature onto at least one plane perpendicular to a plane containing the electrodes.

The application area for these patents is for ECG diagnostics, specifically for in hospital diagnostics.

In contrast the invention herein discloses methods for utilizing an optimal lead set to measure ECG data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Anderson discloses a greater than 20 electrode BSPM vest based system for the estimation of the electrophysiological potentials on the body surface for ECG diagnostics procedures whereas the invention disclosed herein optimal lead set of arbitrary topology including small lead number sets for multiple disease detection,

Selvester, et al. in U.S. Pat. No. 6,947,789 “Method for detecting, sizing and locating old myocardial infarct” discloses a method for detecting and characterizing, in the presence of confounders, a subject's old myocardial infarct (MI) comprising collecting that subject's ECG data from several pre-selected, standard ECG leads, establishing, in the presence of a history of confounding conditions and in relation to selected characteristics of that subject's personal data, such as, interalia, sex, age, and/or race, a set of ECG-data criteria to examine, including R/Q and R/S voltage-amplitude ratio criteria, examining such established criteria set in the context of the mentioned history of confounding conditions, and from said examining, generating an output indicative of the desired detecting and characterizing of an old MI.

The application area for this patent is for ECG diagnostics, specifically for diagnosing “old” myocardial infarcts.

In contrast the invention herein discloses methods for utilizing an optimal lead set to measure ECG data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Selvester discloses a method for collecting a subject's ECG data from several pre-selected, standard ECG leads, and a history of confounding conditions and in relation to selected characteristics of that subject's personal data whereas the invention discloses herein an optimal lead set of arbitrary topology for multiple disease detection and utilizes a matrix method that has disease data embedded within it.

Sheldon, et al. in U.S. Pat. No. 6,937,899 “Ischemia detection” discloses a method for detecting whether a change in the ST segment is accompanied by a corresponding change in the contractility of the heart. Said contractility changes are detected by an accelerometer or pressure sensor and correlated with changes in the ST electrogram segment.

The application area for this patent is for cardiac algorithms used in implantable devices.

In contrast the invention herein discloses methods for utilizing an optimal lead set to measure ECG data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Sheldon discloses a method for detecting whether a change in the ST segment is accompanied by a corresponding change in the contractility of the heart which are detected by an accelerometer or pressure sensor and then correlated with changes in the ST electrogram segment whereas the invention disclosed herein utilizes a body surface optimal lead set of arbitrary topology for multiple disease detection.

Schwartzman, et al. in U.S. Pat. No. 6,725,085 “Method and apparatus for characterizing cardiac tissue from local electrograms” disclose the means to determine the property of cardiac tissue at a local site, a plurality of sites of in a region of a heart may be characterized based on local electrograms measured at the local site, at a plurality of sites or in the region, respectively. The property may be characterized by normalizing the local electrogram, extracting a feature vector from the normalized electrogram, and classifying the tissue property based on the feature vector. The method of may further include computing a map (BSPM) of the tissue property and treating the tissue based on the resultant map. Apparatus to characterize the property includes a catheter and a processor to normalize the local electrogram, extract the feature vector from the electrogram and classify the tissue based on the feature vector. Further a temporal trend based on ST segment changes in a localized ECG provides for a determination that may include computing a normalized value of ST segment changes, and also may include computing characteristics of a body surface spatial distribution of ST segment changes.

The application area for this patent is for ECG systems and sensing catheters used in EP procedures.

In contrast the invention herein discloses methods for utilizing an optimal lead set to measure ECG data, find the measurement extrema over an optimum portion of the Thorax, and to determine probabilities of disease indicators. Further our invention discloses an optimum matrix which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Schwartzman discloses a method for determining a property that may be characterized by normalizing the local electrogram, extracting a feature vector from the normalized electrogram, and classifying the tissue property based on the feature vector and may further include computing a BSPM of the tissue property and treating the tissue based on the resultant map whereas the invention disclosed herein utilizes a optimal body surface lead set of arbitrary topology for multiple disease detection, produces total thoracic and local electrograms, and whose probability of disease conditions is computed based on a set of parameters including ECG measurement extrema and temporal trends.

Albrecht, et al. in U.S. Pat. Nos. 6,047,206 and 5,891,045 “Generation of localized cardiac measures” disclose the means to determine whether the subject is experiencing a myocardial infarction is to capture electrical signals from at least two sensors. The received signals then are processed to obtain a localized cardiac measure that is analyzed to determine said myocardial infarction. A localized cardiac measure is defined as a cardiac measure generated using signals produced by two or more sensors, or electrodes that are spaced by a distance less than the spacing between sensors used to produce standard electrocardiogram leads. Use of the second derivative of the surface potential distribution, the spatial derivative, referred to as the Laplacian ECG, may reflect local activity in discrete regions of the heart.

The application area for this patent is for ECG diagnostics.

In contrast the invention herein discloses methods for utilizing an optimal lead set to measure ECG data, find the measurement extrema over an optimum portion of the Thorax, and to determine probabilities of disease indicators. Further our invention discloses an optimum matrix, which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Albrecht discloses a method for determining a localized cardiac measure is defined as a cardiac measure generated using signals produced by two or more sensors, or electrodes, that are spaced by a distance less than the spacing between sensors used to produce standard electrocardiogram leads whereas the invention disclosed herein utilizes a optimal lead set (topology) for disease detection, and produces total thoracic and local electrograms, and whose probability of disease conditions is computed based on a set of parameters including ECG measurement extrema and temporal trends.

Tereschouk, et al. in U.S. Pat. No. 6,358,214 “ECG scanner” disclose the means to determine an ECG scanner which generates omni directional ECG leads producing tracings that are easier to analyze as they have comparable voltages. The object of this invention is to create an instrument for automatically and systematically synthesizing an array of ECG leads and composing the three-dimensional space in a predetermined order to prevent information loss. Another object of the invention is to develop a method for automatically and systematically analyzing signals in an orderly-synthesized array of ECG leads to detect pathology in a lead that is collinear with a pathological sign. Also the user can use a Trackball to move in 3D.

The application area for this patent is for post capture ECG diagnostics.

In contrast the invention herein discloses methods for utilizing an optimal lead set to measure ECG data, find the measurement extrema over an optimum portion of the Thorax, and to determine probabilities of disease indicators. Further our invention discloses an optimum matrix which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Tereschouk discloses a method for synthesizing an array of ECG leads and composing the three-dimensional space whereas the invention disclosed herein utilizes a optimal lead set (topology) for disease detection.

Brodnick, et al. in U.S. Pat. No. 6,282,440 “Method to identify electrode placement” discloses a method to determine whether the electrodes are incorrectly positioned or are positioned in a non-standard configuration. The method disclosed uses a covariance matrix eigenvector solution that is computed using singular value decomposition (SVD) or Karhunen-Loeve transform (KLT), principal components analysis and principle forces analysis respectively.

The application area for this patent is determine incorrect lead placement.

In contrast the invention herein discloses methods for utilizing an optimal lead set to measure ECG data, find the measurement extrema over an optimum portion of the Thorax, and to determine probabilities of disease indicators. Further our invention discloses an optimum matrix which is computed using source data from a database of 192 lead BSPM ECG data with specified disease set and defined thoracic geometry from a large set of patients. Finally our invention discloses leads systems used for input may be from a few to 100s of uni-polar leads where 4 to 24 leads being typical and output is for 4 to 192 leads where 4 to 32 leads are typical.

In sum Brodnick discloses a method to determine whether the electrodes are incorrectly positioned whereas the invention disclosed herein utilizes a optimal lead set (topology) for disease detection.

Evans, et al. in U.S. Pat. Nos. 5,377,687, 5,318,037 and earlier U.S. Pat. No. 5,161,539 “Method and apparatus for performing mapping-type analysis including use of limited electrode sets” disclose the estimation of a 192 BSPM electrode set from the standard 12 lead set comprised of 10 electrodes. Evans also discloses a coronary disease diagnostic methodology utilizing a probability function to provide a measure of coronary disease likelihood based on 12 basis functions and provide “three or more statistically determined coefficients taken from a set of coefficients to the probability that a patient has a coronary disease”.

The application area for this patent is for ECG diagnostics.

In contrast the invention herein discloses methods for utilizing an optimal lead set to measure ECG data, find the measurement extrema over an optimum portion of the Thorax, and to determine probabilities of disease indicators. Finally our invention discloses leads systems, used for input, may use from a few leads to 100s of uni-polar leads, where 4 to 24 leads are typical, and output is for 4 to 192 leads where 4 to 32 leads are typical. Probability of disease conditions is computed based on a set of parameters including ECG measurement extrema and temporal trends among other parameters.

In sum Evans discloses a method for the estimation of a 192 BSPM electrode set from the standard 12 lead set, which is comprised of 10 electrodes, whereas the invention disclosed herein utilizes a optimal lead set (topology) that can utilize a wide variety of lead number and topologies. Further Evans also discloses a coronary disease diagnostic methodology utilizing a probability function to provide a measure of coronary disease likelihood where Evan's “statistically determined coefficients” provide his “twelve basis functions” whereas the invention disclosed herein utilizes a wide spectrum of parameters, including ECG measurement extrema, and temporal trends among them.

### OTHER REFERENCES

Optimal Left Ventricular Hypertrophy Classification and Quantification: Insights from Body Surface Potential Maps

Fred Kornreich, Bellingen, Belgium

International Journal of Bioelectromagnetism

Vol. 5, No. 1, 2003, pp. 197-198

Body surface mapping improves early diagnosis of acute myocardial infarction in patients with chest pain and left bundle branch block

S J Maynard, I B A Menown, G Manoharan, J Allen, J McC Anderson, A A J Adgey . . .

Heart 2003;89:998-1002

Clinically Practical Lead Systems for Improved Electrocardiography: Comparison with Precordial Grids and Conventional Lead Systems

Robert L. Lux, Ph.D., Mary Jo Burgess, M.D., Roland F. Wyatt, B. S., A. Kerry Evans, M.E., G. Michael Vincent, M.D., And J. A. Abildskov, M.D.

Circulation Vol 59, No 2, February 1979, Page 360

Electrocardiographic potential correlations: rationale and basis for lead selection and ECG estimation.

Robert L. Lux

Nora Eccles Harrison Cardiovascular Research and Training Institute, University of Utah, Salt Lake City 84112-5000, USA

Errors in Electrocardiographic Parameter Estimation from Standard Leadsets

Robert S. MacLeod, Robert L. Lux

J. Electrocardiol. Vol. 28, Suppl., pages 98â [symbol] ″103, 1995

Oct. 14, 2001

Optimal 5 Lead ECG System

Robert L. Lux, PhD

Matryx Group, Inc.—Internal Research

Nov. 3, 2005

### BRIEF SUMMARY OF THE INVENTION

The invention disclosed herein methods for utilizing an optimal lead set to measure electrocardiographic data, find the measurement extrema over an optimum portion of the Thorax, and to determine probabilities of cardiac disease indicators.

The optimal lead topology disclosed is designed to produce estimates of total thoracic electrocardiographic information, low noise and errors within the constraints imposed by the measured leads' associated constraint set, which include disease targets. Importantly an optimal electrocardiographic topology and measured lead set is deemed optimal when the estimated ECG lead topology provides the lowest global estimation errors.

An “optimal” lead system is one that provides the best cardiac disease detection with the fewest number of electrodes. Since there is an “information structure” which depends on the correlation of ECG information between all pairs of sites on the body surface, it is possible to determine such an optimal lead system, as well as an estimation transformation that estimates all the information at un-sampled locations.

An optimum electrode topology is one that places unipolar electrodes on the Thorax in arbitrary positions and not in a grid like manner (such as used by a BSPM {Body Surface Potential Map} vest) nor necessarily in those positions used in current practice such as for standard 12 lead or EASI leads.

The optimum matrix is computed using source data from a database of 192 unipolar lead BSPM electrocardiographic data, providing specified disease set and defined thoracic geometry data, from a large set of patients.

The derived optimum covariance matrix has the patient data subsumed within the matrix rather than as a separate and potentially large database.

The performance from an optimum lead system provides the following benefits:
Provides better estimates of total thoracic ECG information,
Greater signal to noise ratio vs. 12-lead and other reduced lead systems.
Transient ischemia may see and increased detection sensitivity of 10-15%,
Improved arrhythmia detection & classification via:
   Increased S.N.R. of P-wave signal capture (for atrial activity)
   Improved capture of QRS, ST-T and delta waves.
Improved ST-T wave signal-to-noise (for ischemia),
Improved capture of T wave morphology (for T wave alternans)

The accuracy performance for an optimum lead system increases with the total number of measure leads but the accuracy increase begins to “flatten” at 16 leads and is totally flat at 32 leads. The invention described herein utilizes a measured lead set that has been optimized for a variety of constraints. One constraint is the total number of leads. The total number of leads may be constrained for the reasons of practical lead placement, cost of electrodes, ease of application and patient comfort.

Lead systems used for input may utilize from a few leads to 100s of unipolar leads where 4 to 24 leads is typical and where output leads may be from 4 to 192 leads, but 4 to 32 leads is typical.

Other systems may estimate leads from the standard 12 lead system and these methods provide poor performance in that, for example, Ischemia extrema may be located in regions not detected or even hinted at by the 12 leads including for the typical I, II, aVf leads. Note that the important point here is that a full BSPM 192 lead set, for example, can see distributions which are below and beyond “clinical threshold” for ST changes and therefore are not detected or even hinted at when using the standard 12 lead ECG.

The invention also produces total thoracic, local and standard lead electrograms (ECG waveforms) such as the standard 12 lead system.

The probability of disease conditions is computed based on a set of parameters, especially electrocardiographic measurement extrema and temporal trends thereof, and these methods perform without the need of specific patient history.

### BRIEF DESCRIPTION OF THE DRAWINGS

FIG. 1—System Diagram

FIG. 2—Analysis process

FIG. 3—Sensor Harness Topology

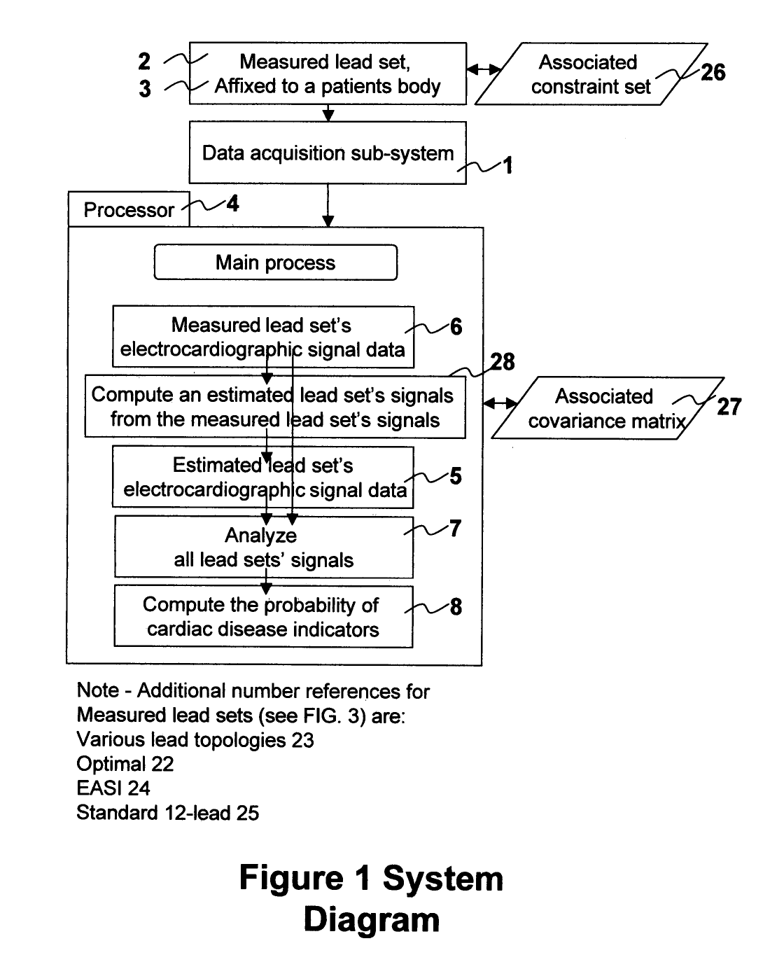

FIG. 1 System Diagram

Electrocardiographic data is acquired from the measured lead set and measurements are made.

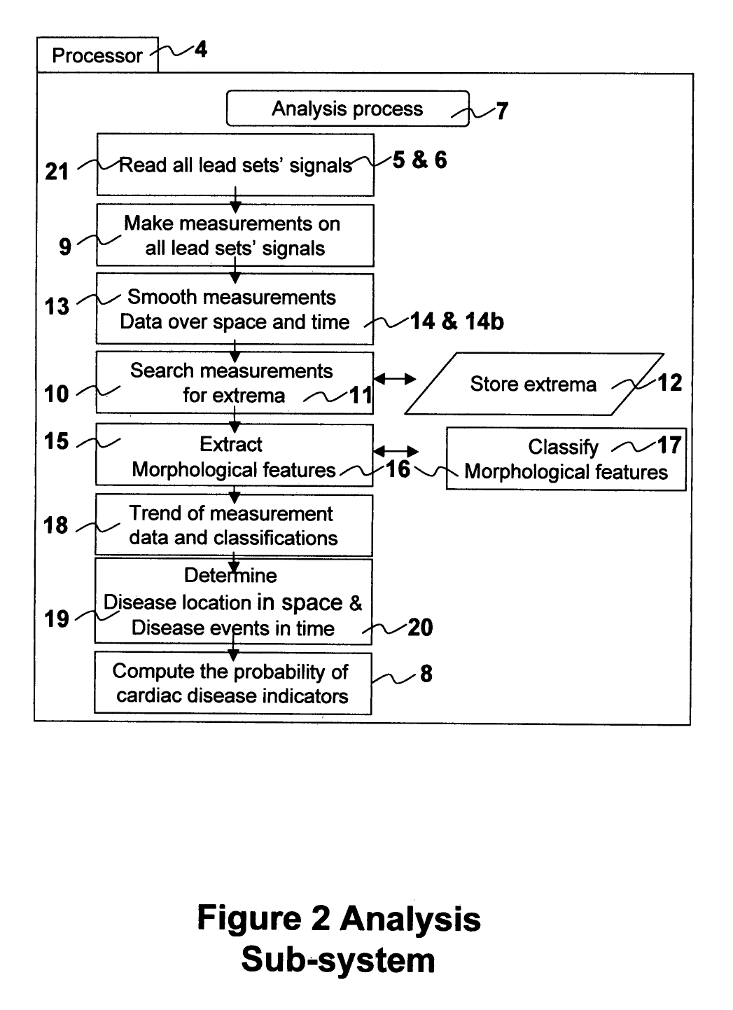

FIG. 2 Analysis Process

Electrocardiographic measurements are analyzed and disease probabilities computed.

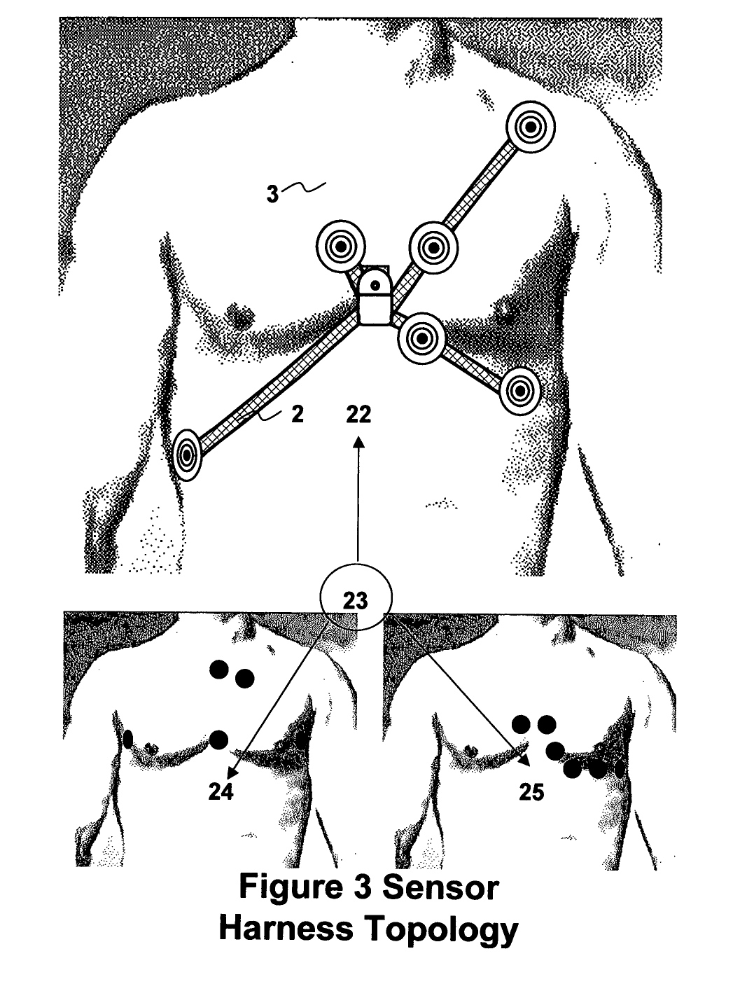

FIG. 3 Sensor Harness Topology

An ECG sensor harness supporting an Optimal for Ischemia-5 lead electrode topology affixed on a patient's body.

### REFERENCE NUMERALS IN DRAWINGS

FIG. 1 System Diagram

Data acquisition sub-system 1

Measured lead set 2 (see FIG. 3)

Associated constraint set 26

Affixed to a patient's body 3 (see FIG. 3)

Processor 4

Estimated lead set's signals 5

Measured lead set's signals 6

Analyzes all lead set's signals 7 (see FIG. 2)

Computes the probability of cardiac disease indicators 8

Computation of the estimated lead set's signals 28

Associated covariance matrix 27

FIG. 2 Analysis Process

Analysis process 7

All the lead sets' signals 5 & 6

Read all the lead sets' signals 21

Make measurements on the electrocardiographic data 9

Search the electrocardiographic measurements 10

Extrema 11

Store the extrema 12

Smooth the electrocardiographic measurement data 13

Smooth the electrocardiographic measurement data over space 14 and time 14b

Extract morphological features 15

Morphological features 16

Classify morphological features 17

Trend measurement data and classifications 18

Determine disease location 19

Determine disease events in space and time 20

Compute cardiac disease indicators 8

FIG. 3 Sensor Harness Topology

Measured lead set 2

Patients body 3

Electrode topology 23

Each of the lead sets discussed 23

Optimum 5-lead for ischemia 22

EASI-4 lead 24

Standard 12-lead—Precordials only 25

### DETAILED DESCRIPTION OF THE INVENTION

Referring to FIG. 1 the system for computing cardiac disease indicators, utilizes a data acquisition sub-system 1 for capturing electrocardiographic unipolar (ECG) signal data from a measured lead set 2—FIG. 3, and affixed to a patients body 3, where a processor 4 continuously computes 28 an estimated lead set's signals 5 from the measured lead set's signals 6 and then analyzes all lead sets' signals 7 and finally computes the probability of cardiac disease indicators 8.

Referring to FIG. 2 the analysis process 7 computes cardiac disease indicators 8 and disease location 19 in the following process steps:

read 21 all the lead sets' signals 5 & 6;

make measurements 9 on all the lead sets' signals 5 & 6;

smooth 13 the measurement data 9 over space 14 and time 14b;

search 10 the measurements for extrema 11 and store the extrema 12; extract 15 morphological features 16 and classify 17 the morphological features;

trend 18 the measurement data and classifications;

determine disease location 19 and events 20 in space and time; and

compute cardiac disease indicators 8.

Measurements are performed on both the measured lead 6 set's signals and the estimated 5 signals' measurements include ST-T, delta wave, QRS, P-wave, T-wave, alternans, and confounders among any other measurement types.

Spatial and temporal filter functions are used to smooth 13 the measurement data over space and time.

Search 10 is performed for maxima and minima over all the measurements in space and time.

Extraction 15 and classification 17 of morphological features 16 is performed by statistically matching the measurements and eigenvalues against a set of disease-identified measurements and eigenvalues.

Trending 18 of the measurement data and classifications is provided by determining the temporal derivative of any dynamic variable especially measurements, eigenvalues, classification, events, and alarms. Secondly, dynamic rules of classification are applied recursively thereby providing further temporal derivative data.

Disease location 19 is performed by search of classification, trending, and disease probability in the data store in order to determine the location of the diseases spatially located on the thorax, or epicardial heart surface. Further the temporal location of disease events 20 is determined.

Cardiac disease probabilities 8 are determined for the common cardiac disease conditions such as arrhythmia, ischemia, and myocardial infarction.

Computation of cardiac disease probabilities 8 is based on a statistical process that utilizes parameters such as measurements, eigenvalues, classification, events, and alarms.

Both static and dynamic (trend) parameters are used calculate disease probability.

The measured lead set 6 is defined, as an electrode set whose ECG electrode topology 23—FIG. 3 is spatially defined over the patient's thorax.

A measured lead set's topology is defined using the 192 lead BSPM scheme. Importantly one optimal lead set, which has been optimized for ischemia detection using 5 leads, is defined as: 140, 112, 133, 104, 88, where 54 is a reference lead, see 22.

In comparison the standard 12 lead, which is un-optimized, is defined as: V1-88, V2-100, V3-113, V4-126, V5-138, V6-150 plus limb leads, see 25.

Also the un-optimized EASI-4 lead is defined as: 54, 85, 102, 138 plus reference lead, see 24.

The computation of the estimated lead set's signals 28 from the measured lead set's signals uses the following process steps:
1) select an optimal lead topology 22, which has associated a constraint set 26 and a covariance matrix 27;
2) affix the selected lead topology 23 to the patient's body 3, which provides the measured lead set 6.
3) load the associated covariance matrix 27 into the processor 4;
4) acquire the electrocardiographic signals 1 from the measured topology lead set 5;
5) calculate the estimated signals 28 using matrix multiplication of the measured lead set signals by the associated covariance matrix 27; and
6) store said estimated set signals 5 together with the measured lead set signals 6.

An optimal lead set is one found to achieve optimum estimates of total thoracic electrocardiographic information, low noise and estimation errors while adhering to the constraints imposed by the measured lead's associated constraint set 26. Therefore an optimal lead topology and measured lead set is logically optimal when the candidate topology performs at the lowest noise and estimation errors. An associated constraint set 26 is used to define and compute the optimal electrocardiographic topology. The following are the main, but not exclusive, set of constraints;
number and topology of measured leads,
number and topology of estimated leads,
standard leads—number and location,
practical leads—number and location,
additional electrodes as required for physician directed electrode placement,
difficulty of lead placement on a patient body shape,
performance impact of misplaced or dropped leads,
patient body size and shape types,
patient independent,
disease targets and measurements including ischemia, ST-T, arrhythmia,
P-wave, T-wave, alternans, confounders and combinations thereof,
localization capability for Ischemia, myocardial infarction, and arrhythmia,
patient compliance,
design viability of an electrode support harness.

What is claimed is:

1. A method to determine cardiac disease indicators, the method comprising:
acquiring electrocardiographic signals from a measured lead set affixed to a patients body;
computing an estimated lead set's signals from the measured lead set's signals; continuously analyzing all lead set's signals; and
computing probability of said cardiac disease indicators,
wherein said measured lead set is an optimal electrode topology and is configured to provide optimum estimates of total thoracic electrocardiographic information, and low noise and errors within the constraints imposed by the associated constraint set.

2. The method of claim 1, wherein said cardiac disease indicators are determined by the following said continuous analysis steps:
measuring electrocardiographic data of all said lead set's signals;
searching said electrocardiographic measurements for extrema and storing said extrema;
smoothing said electrocardiographic measurement data over space and time;
identifying morphological features and classifying said morphological features;
trending said measurement data and classifications;
computing said cardiac disease probabilities; and
determining disease location and events in space and time.

3. The method of claim 2, wherein the method for said searching measurement for extrema is comprised of performing ECG measurements, storing said measurements, and searching said measurements for maximum and minimums.

4. The method of claim 2, wherein the method for said smoothing measurement data over space and time is comprised of invoking spatial and temporal filter functions on said measurements thereby providing smoothed ECG data over space and time.

5. The method of claim 2, wherein the method for said classifying is comprised of statistically matching said electrocardiographic measurements and eigenvalues against a set of disease-identified electrocardiographic measurements and eigenvalues.

6. The method of claim 2, wherein the method for said trending is comprised of the steps;
determining the temporal derivative of any dynamic variable from the group consisting of, but not limited to: electrocardiographic measurements, eigenvalues, classification, events, alarms; and
recursively applying additional dynamic rules of classification thereof to said temporal derivative data.

7. The method of claim 2, wherein said cardiac disease probabilities are for disease conditions from the group consisting of, but not limited to: arrhythmia, ischemia, myocardial infarction.

8. The method of claim 2, wherein the method for said disease location is comprised of searching the classification, trending, and disease probability data store in order to determine the location of said diseases diagnosed spatially located on the thorax, or epicardial heart surface, as well as the temporal location of cardiac events.

9. The method of claim 1, wherein said measured lead set is the measured electrode set whose said electrode topology is spatially defined over said Thorax.

10. The method of claim 1, wherein said measured lead set is comprised of the following lead sets, whose topologies are numbered using the 192 lead BSPM scheme, from the group consisting of, but not limited to:
Optimized for Ischemia and 5 leads—140, 112, 133, 104, 88, 54—Reference;
Un-optimized standard-12 leads—V1-88, V2-100, V3-113, V4-126, V5-138, V6-150 plus limb leads;
Un-optimized EASI-4 leads: 54, 85, 102, 138 plus reference lead.

11. The method of claim 1, wherein said computing of an estimated lead set's signals from a measured lead set's signals, is comprised of the following steps:
selecting said optimal lead topology whose said measured lead set is affixed on the patient's body, wherein said optimal topology has an associated said constraint set and covariance matrix;
loading said associated covariance matrix;
acquiring electrocardiographic signals from said measured lead set;
calculating said estimated electrocardiographic signals using matrix multiplication of said measured lead set signals by said associated covariance matrix; and
storing said estimated lead set signals together with said measured lead set signals.

12. The method of claim 1, wherein said associated constraint set is used to define and compute said optimal electrode topology, from the group, and in any combination but not limited to, the following said constraints;
number and topology of measured leads,
number and topology of estimated leads,
standard leads—number and location,
practical leads—number and location,
additional electrodes as required for physician directed electrode placement,
difficulty of lead placement on a patient body shape,
performance impact of misplaced or dropped leads,
patient body size and shape types,
patient independent optimization,
disease targets and measurements including Ischemia, ST-T, Arrhythmia,
P-wave, T-wave, alternans, confounders and combinations thereof,
localization capability for Ischemia, Myocardial Infarction, and Arrhythmia,
patient compliance,
design viability of an electrode support harness.

13. The method of claim 1, wherein said optimal electrode topology and measured lead set is optimal when said topology performs at the lowest global estimation errors, for said estimated lead set.

14. The method of claim 2, wherein said computing of cardiac disease probabilities is comprised of a statistical process that utilizes parameters from the group consisting of, but not limited to: electrocardiographic measurements, eigenvalues, classification, events, alarms; and utilizes both said parameters and said trending of said parameters to calculate disease probability.

15. A system for computing cardiac disease indicators, comprising:
a data acquisition sub-system acquire electrocardiographic signal data;
a measured lead set affixed to a patients body configured by the constraints imposed by the measured lead's associated constraint set which achieves optimum estimates of total thoracic electrocardiographic information, low noise and estimation errors;
a processor that continuously:
(1) computes an estimated lead set's signals from the measured lead set's signals;
(2) analyzes all lead sets' signals; and
(3) computes the probability of said cardiac disease indicators.

16. The cardiac disease indicator system of claim 15, wherein a continuous analysis process computes said cardiac disease indicators:
measure electrocardiographic data of all said lead set's signals;
search said measurements for extrema and store said extrema;
smooth said measurement data over space and time;
extract morphological features and classify said morphological features;
trend said measurement data and classifications;
compute said cardiac disease probabilities; and
determine disease location and events in space and time.

17. The continuous analysis process of claim 16, wherein the process for said search measurement for extrema makes electrocardiographic measurements, store said measurements, and search said measurements for maxima and minima.

18. The continuous analysis process of claim 16, wherein the process for said smooth measurement data over space and time invoke spatial and temporal filter functions on said electrocardiographic measurement data thereby providing smoothed electrocardiographic data over space and time.

19. The continuous analysis process of claim 16, wherein the process for said classify morphological features statistically matches said measurements and eigenvalues against a set of disease-identified electrocardiographic measurements and eigenvalues.

20. The continuous analysis process of claim 16, wherein the process for said trend measurement data and classifications, comprising;
determine the temporal derivative of any dynamic variable from the group consisting of, but not limited to: electrocardiographic measurements, eigenvalues, classification, events, alarms; and
apply, recursively, additional dynamic rules of classification thereof to said temporal derivative data.

21. The continuous analysis process of claim 16, wherein said cardiac disease probabilities are for disease conditions from the group consisting of, but not limited to: Arrhythmia, Ischemia, Myocardial Infarction.

22. The continuous analysis process of claim 16, wherein the process for said disease location is comprised of said search of classification, trending, and disease probability data store to determine the location of said diseases diagnosed spatially located on the Thorax, or Epicardial heart surface, as well as the temporal location of said disease events.

23. The cardiac disease indicator system of claim 15, wherein said measured lead set is the measured electrode set whose said electrode topology is spatially defined over said Thorax.

24. The cardiac disease indicator system of claim 15, wherein said measured lead set is comprised from the following lead sets, whose topologies are defined using the 192 lead BSPM scheme, from the group consisting of, but not limited to: optimized for Ischemia and 5 leads—140, 112, 133, 104, 88, 54—Reference;
un-optimized standard-12 leads—V1-88, V2-100, V3-113, V4-126, V5-138, V6-150 plus limb leads;
un-optimized EASI-4 leads: 54, 85, 102, 138 plus reference lead.

25. The cardiac disease indicator system of claim 15, wherein said computation of an estimated lead set's signals from the measured lead set's signals, is comprised of the following process steps:
selection of said optimal lead topology whose said measured lead set is affixed on to the patient's body, wherein said optimal topology has an associated said constraint set and covariance matrix;
load said associated covariance matrix;
acquire electrocardiographic signals from said measured electrode topology lead set;
calculate said estimated electrocardiographic signals using matrix multiplication of said measured lead set signals by said associated covariance matrix; and
store said estimated electrocardiographic set signals together with said measured lead set signals.

26. The cardiac disease indicator system of claim 15, wherein said associated constraint set is used to define and compute said optimal lead topology, from the group, and in any combination but not limited to, the following said constraints;
number and topology of measured leads,
number and topology of estimated leads,
standard leads—number and location,
practical leads—number and location,
additional electrodes as required for physician directed electrode placement,
difficulty of lead placement on a patient body shape,
performance impact of misplaced or dropped leads,
patient body size and shape types,
patient independent optimization,
disease targets and measurements including Ischemia, ST-T, Arrhythmia, P-wave, T-wave, alternans, confounders and combinations thereof,
localization capability for Ischemia, Myocardial Infarction, and Arrhythmia,
patient compliance,
design viability of an electrode support system.

27. The cardiac disease indicator system of claim 15, wherein said optimal lead topology and measured lead set is logically optimal when said topology performs said lowest noise and estimation errors, for said estimated lead set.

28. The continuous analysis process of claim 16, wherein said computation of cardiac disease probabilities is comprised of a statistical process that utilizes parameters from the group consisting of, but not limited to: electrocardiographic measurements, eigenvalues, classification, events, alarms; and utilizes both said parameters and said trend of said parameters to calculate disease probability.
